(19) United States
(12) Patent Application Publication
(10) Pub. No.: US 2014/0052036 A1
(43) Pub. Date: Feb. 20, 2014

(54) Back brace frame with one power module

(71) Applicant: Dae Shik Park, Rowland Heights, CA (US)

(72) Inventor: Dae Shik Park, Rowland Heights, CA (US)

(21) Appl. No.: 13/573,058

(22) Filed: Aug. 17, 2012

(51) Int. Cl. A61F 5/01

(52) U.S. Cl. 602/5

(57) ABSTRACT

A waist supporting back brace frame for a detachment type waist protecting belt to hold the vertebra region of a vertebra related patient is provided. The frame is comprised of two back rest frames, one power module, one puller, one string that is engaged and connects two parts (left and right) of the power module, and two set of broad bands. Each part of the power module is equipped with four pairs of string conveying mini rollers. Pluralities of gear teeth are developed on the surface of the string conveying mini rollers along the vertical axis thereof. Upper and lower corner of the teeth is rounded to minimize friction between the roller and the power module when a wearer put on the frame and fasten.

### FIELD OF THE INVENTION

The present invention relates to a waist supporting back brace frame, more particularly, a frame enabling self-adjustment of the waist support frame to fit to the curvatures of a patient's with only one hand, especially fastening the strings evenly throughout the frame to apply even pressure to the body of the wearer along the whole portion supported by the frame.

### BACKGROUND OF THE INVENTION

As is well known in the industry, the waist-protecting belt, known as an abdominal support or a pelvic girdle, is comprised of a resilient belt made of a proper material such as spandex, and a rigid back supporting plate. The role of a waist-protecting belt is to slightly compress and support the waist of a vertebra related patient's body. This prevents pain by keeping the waist in straight vertical alignment. Adjusting the resilient belt controls pressure to the patient's back and body. However, the back supporting plate or frame, which directly touches the patient's back, is usually made of one rigid plate made of plastic or gypsum. Therefore, if the back supporting plate does not match the shape of the patient's back, it often causes undesired pain to the patient. A time consuming process of patterning the contour of a patient's back is necessary, to make the back supporting plate more effectively fit the curvature of the patient's back. U.S. Pat. No. 7,905,849 of the current application provided a back brace frame that resolves all the previous drawbacks and especially provide a back brace frame that can be used by a patient whose one hand is not available. However, users complain that it is very hard to fasten the frame properly to press the vertebra region uniformly because the pressure on the center part of the frame is too much strong when the user fasten the frame tightly to feel some pressure on the upper or lower part of the frame. So, it is purpose of the current application to provide a back brace frame that retains advantages of the back brace frame disclosed in the U.S. Pat. No. 7,905,849 and has another advantage of fastening the back brace frame evenly through out the frame to press the wearer's vertebra region with even pressure.

### DESCRIPTION OF THE PRIOR ARTS

U.S. Pat. No. 7,905,849 to Park illustrates a waist support frame for a detachment type waist protecting belt to hold the vertebra region of a vertebra related patient, which is comprised of two back rest frames, two Y-shape hinges, two power modules, two pullers, two sets of strings each of them are engaged and connects on one part (upper- or lower-part) of the two power modules, and two set of broad bands. Each power module is equipped with twelve pair of pulling code conveying mini rollers. String arrangement through these twelve pair of rollers enables the inventor to provide a waist support frame that can be used by a user whose one hand is not available without rotating the frame around the user's body with only one hand. U.S. Pat. No. 7,083,585 to Latham illustrates a string arrangement for a detachment type waist-protecting belt to hold the vertebra region of a vertebra related patient is provided. U.S. Pat. No. 6,951,547 to Park et al. illustrates a waist support frame for a detachment type waist protecting belt to hold the vertebra region of a vertebra related patient is provided. U.S. Pat. No. 6,322,529 to Chung illustrates a detachment type waist-protecting belt including a waist support, which fits a contour of the waist of the human body. The support is one-piece of solid material. U.S. Pat. No. 5,399,150 Saunders illustrates a lumbosacral back support band provided with a releasable attaching back support system, which is made of one piece of composite band. U.S. Pat. No. 5,195,948 to Hill, et al. illustrates a back support device comprised of a belt structure designed to fit substantially around the waist of a user. An inflatable air bladder is attached inside the belt structure so that it is positioned adjacent to the lower back when the back support device is worn. U.S. Pat. No. 5,127,897 to Roller illustrates a therapeutic back support device including a plastic back support plate, which is coupled to a human body to forwardly direct the plate. U.S. Pat. No. 4,752,982 to Jones, et al. illustrates an adjustable back support apparatus with an anchor assembly to adjustably connect the main support, and a cushion assembly connected to the main support base assembly. U.S. Pat. No. 3,889,664 to Heuser, et al. illustrates two torso belt members, joined together with a jack screw connector, intended to apply traction to the user between the pair of belts.

None of the prior arts introduces a back support plate, the shape of which is easily adjustable with the adjustment of a single strap even by a handicapped user and holding the fastening strings at its fastened position.

### SUMMARY OF THE INVENTION

As is well known in the industry, the waist-protecting belt, known as an abdominal support or a pelvic girdle, is comprised of a resilient belt made of a proper material such as spandex, and a rigid back supporting plate. The role of a waist-protecting belt is to slightly compress and support the waist of a vertebra related patient's body. This prevents pain by keeping the waist in straight vertical alignment. Adjusting the resilient belt controls pressure to the patient's back and body. However, the back supporting plate or frame, which directly touches the patient's back, is usually made of one rigid plate made of plastic or gypsum. Therefore, if the back supporting plate does not match the shape of the patient's back, it often causes undesired pain to the patient. A time consuming process of patterning the contour of a patient's back is necessary, to make the back supporting plate more effectively fit the curvature of the patient's back. In the U.S. Pat. No. 7,905,849 granted the current applicant, a waist support frame for a detachment type waist protecting belt to hold the vertebra region of a vertebra related patient is provided. The frame is comprised of two back rest frames, two Y-shape hinges, two power modules, two pullers, two sets of strings each of them are engaged and connects on one part (upper- or lower-part) of the two power modules, and two set of broad bands. Each power module is equipped with twelve pair of pulling code conveying mini rollers. String arrangement through these twelve pair of rollers enables the inventor to provide a waist support frame that can be used by a user whose one hand is not available without rotating the frame around the user's body with only one hand. But, from marketing of the sample of the patent '849, which is grated to current applicant, it is found that the fastening string is not evenly fastened. Therefore, portions of frame that are away from the center is not fastened enough and as a result the pressure applied to the wearer's body is not uniform. So, when a wearer needs a little bit more pressure on some portion of the back he/she fasten the frame more tighter to feel some pressure on a specific portion of the back. But, due to the uneven fastening unnecessary hard pressure is applied on the center portion of the frame. Then the fastening ring may be disconnected or the center of the back portion of the wearer's body may be exposed to unnecessary hard pressure. It is undesirable situation for a wearer when he/she is recovering from a back surgery. So, it is the purpose of the current application to provide a back supporting frame that retains all the advantage of the applicant's previous invention while tightening the frame evenly across the whole portion thereof.

### BRIEF DESCRIPTION OF THE DRAWINGS

FIG. 1 is an enlarged view of an indivisual power module showing the string arrangement that enables a handicapped user fasten the back brace without rotating of the back brace along the body of the user with one hand.

FIG. 2 is a detailed view of the pulling cord conveys mini roller of current application.

FIG. 3 is a front view of the back brace frame with one individual power module according to current application.

### DETAILED DESCRIPTION OF THE PREFERRED EMBODIMENT

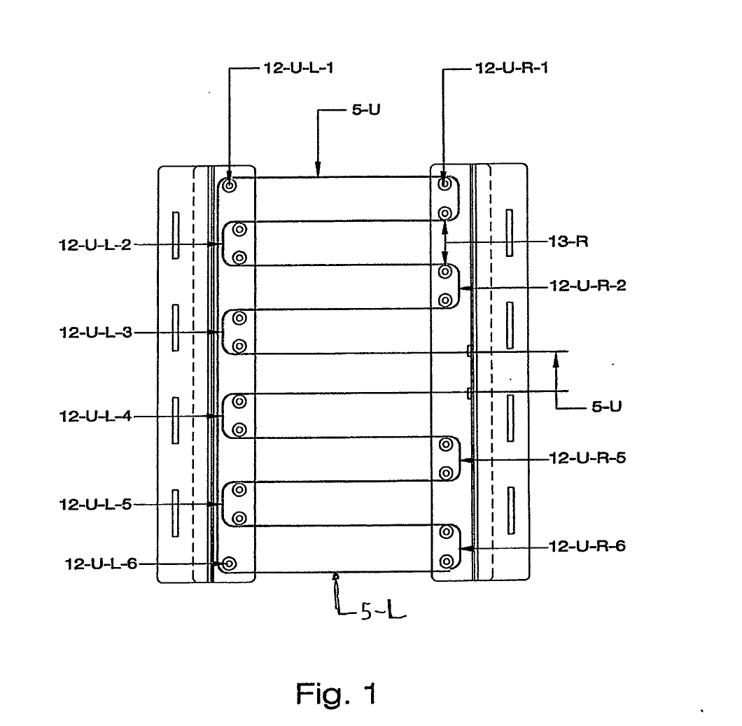

FIG. 1 is an enlarged view of an indivisual power module (3) showing string's arrangement that enables a handicapped user fasten the back brace without rotating of the back brace along the body of the user with one hand. This view is seen when both of the plastic covers (11), that covers the string (5) and pulling code conveying mini rollers (12), are removed from the plates. One half part of the string (5) that passes through the mini rollers (12) locate on the upper half part of the power module (3) is named as (5-U) and the other half part of the string (5) that passes through the mini rollers (12) locate on the lower part of the power module is named as (5-L).

A single string (5) is engaged through 12 pairs of pulling code conveying mini rollers (12). The six pair of rollers, roller sets, installed on the upper plate of left side (7-U-L) is numbered as (12-U-L-1), (12-U-L-2), (12-U-L-3), (12-U-L-4), (12-U-L-5), and (12-U-L-6). The other six pairs of the rollers installed on the upper plate of right side (7-U-R) are numbered as (12-U-R-1), (12-U-R-2), (12-U-R-3), (12-U-R-4), (12-U-R-5), and (12-U-R-6). On the upper plate of left side (7-U-L), two roller sets (12-U-L-1) and (12-U-L-6), which constitute the upper most and lower most one, are located more left than the other four roller sets of (12-U-L-2), (12-U-L-3), (12-U-L-4) and (12-U-L-5). Those four roller sets (12-L-U-2) to (12-L-U-5) align in a row with same intervals (13-L).

Meanwhile, on the upper plate of right side (7-U-R), 6 roller sets align in a row. The upper two roller sets of (12-U-R-1), (12-U-R-2) and the lower two roller sets of (12-U-R-5)) and (12-U-R-5) are spaced with same interval (13-R) that is the same as the interval (13-L) on the left plate. Two roller sets of (12-U-R-3) and (12-U-R-4) that locate at the center has different interval (13-R′) between them.

The center portion of the string (5) passes through between the two roller of the roller set (12-U-L-1) and the two rollers of the roller set (12-U-L-6).

One side of the string (5) that passes through between the two rollers of the roller set (12-U-L-1) go around the roller set of (12-U-R-1) on the plate of right side (7-U-R) and then return to the plate of left side (7-U-L) to go around the roller set of (12-U-L-2). This portion of the string is names as upper string (5-U). The upper string (5-U) keep on go around the roller sets of (12-U-R-2) and (12-U-L-3) in series and finally pass through between the two rollers of roller set (12-U-R-3).

The other side of the string (5) that passes through between the two rollers of the roller set (12-U-L-6) go around the roller set (12-U-R-6) on the plate of right side (7-U-R) and then return to the plate of left side (7-U-L) to go around the roller set of (12-U-L-5). This portion of the string is names as lower string (5-L). The lower string (5-L) keep on go around the roller sets of (12-U-R-5) and (12-U-L-4) in series and finally pass through between the two rollers (12) of roller set (12-U-R-4).

From the marketing of the back brace of the U.S. Pat. No. 7,905,849, it was found that a wearer feels the pressure on his/her back is not uniform. The applicant investigated the reason and found that the string (5) does not fasten the back brace evenly through out the power module (3) when wear the back brace and fasten the string (5). Even though the applicant considered the friction between the roller (12) and the plastic cover (11) and selected materials, the friction increases when the back brace is worn and as a result the power module (3) is bent along the body line of the wearer.

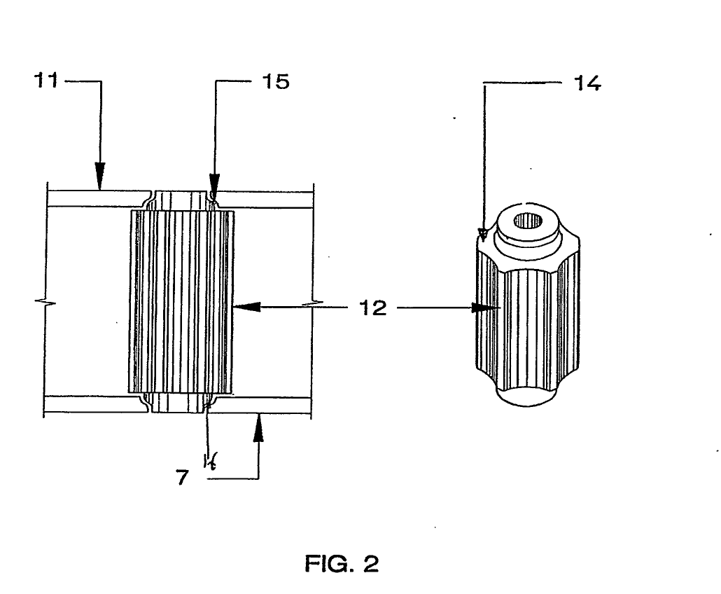

So, the applicant designed a new mini roller (12) as shown in the FIG. 2, which is a detailed view of the pulling cord conveys mini roller (12) of current application. Compared to the previous roller of U.S. Pat. No. 7,905,849, the mini roller (12) of the current application has pluralities of gear teeth (14) developed on the surface of the mini roller (12) along the vertical axis thereof. Another feature of the new mini roller (12) is that the upper corner (15) and lower corner (16) of the teeth (14) are rounded to avoid additional friction even the back brace plate (7) and the plastic cover (11) are bent to new positions. The rounded corners, (16) and (17), prevent direct contact of the teeth (14) and the back brace plate (7) and the plastic cover (11) to minimize the friction between them.

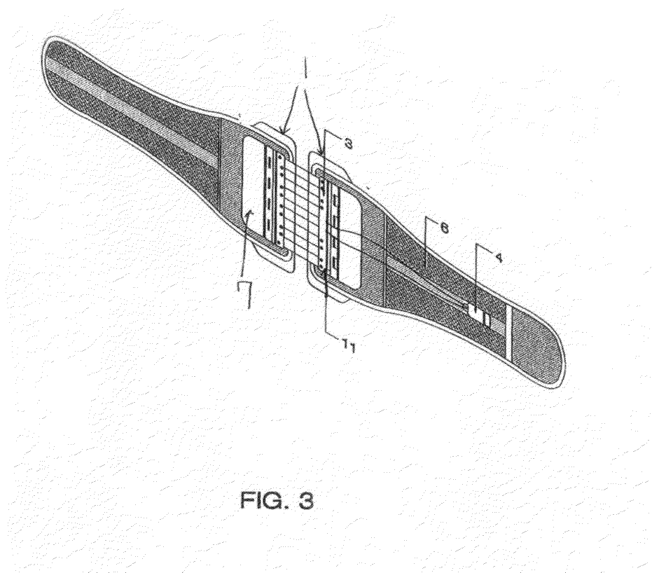

FIG. 3 is a front view of the back brace frame with one individual power module (3) according to current application. The back brace frame with one individual power module according to currenst application is comprised of two back rest frames (1), one power module (3), one puller (4), one set of string (5) that is engaged and connects the two parts of the power module (3), and two set of broad bands (6). This arrangement is for a patient who can use only one hand. Referring to FIG. 1 and FIG. 3, when a user pull the puller (4) to fasten the frame, the tension that is applied on the upper string (5-U) of the portion between the roller sets of (12-U-R-3) and (12-U-L-3) and on the lower string (5-L) of the portion between the roller sets of (12-U-R-4) and (12-U-L-4) is the maximum. If there is no friction at all caused by the roller sets, the tension applied on the string (5) of the portion between the two pair of roller sets of (12-U-R-3), (12-U-L-3) and (12-U-R-4), (12-U-L-4) will be the same as the tension that is applied between the most remote roller sets of (12-U-L-1) and (12-U-L-6).

However, when a user wears the back brace frame, the back rest frames (1) and the power module (3), both of which are made of stiff but bendable plastic, is adjusted to fit the wearer's body line. Then the back brace plate (7) and the plastic cover (11) are bent to one direction as shown in the FIG. 3. In such situation, when the mini roller is in the shape of the mini roller of the previous patent of U.S. Pat. No. 7,905,849, the upper and lower corner of the roller will contact with the back brace plate (7) and the plastic cover (11). Then the mini rollers of the '849 can not roll by the friction created by the rollers and the string. As a result, the tension applied to the string between the roller sets of (12-U-L-1) and (12-U-L-6) will be much lower than the tension applied to the string (5-U) between the roller sets of (12-U-R-3) and (12-U-L-3) and the tension of the string (5-L) between the roller sets of (12-U-R-4) and (12-U-L-4) due to the frictions created by the roller sets of (12-U-L-1), (12-U-L-2), (12-U-L-3), (12-U-L-4), (12-U-L-5), (12-U-L-6), (12-U-R-1), (12-U-R-2), (12-U-R-3), (12-U-R-4), (12-U-R-5), and (12-U-R-6).

However, since the new mini rollers (12) in the current applicant has pluralities of gear teeth (14) developed on the surface of the mini roller (12) along the vertical axis thereof, the gear teeth (14) contacts with the string (5) to rotate the mini rollers (12) easily. In addition to that the upper corner (15) and lower corner (16) of the teeth (14) are rounded to avoid additional friction even the back brace plate (7) and the plastic cover (11) are bent. The rounded corners, (15) and (16), prevent direct contact of the gear teeth (14) and the back brace plate (7) and the plastic cover (11) to minimize the friction between them. Due to the combined effect of the gear teeth (14) and the rounded upper and lower corner of the teeth (14), it is easier to fasten the back brace frame with evenly distributed fastening force.

For example, when a user wears the back brace frame and pulls the puller (4), the upper string (5-U) and lower string (5-L) are pulled and rotates the pair of mini rollers of (12-U-R-3) and (12-U-R-4) respectively according to the friction between the gear teeth (14) of the mini rollers of (12-U-R-3) and (12-U-R-4) and each string (5-U) and (5-L). Then the gear teeth (14) of the mini rollers of (12-U-R-3) pulls the next portion of the upper string (5-U) between mini rollers of (12-U-R-3) and (12-U-L-3). At the same time the gear teeth (14) of the mini rollers of (12-U-R-4) pulls the next portion of the string (5-L) between the rollers of and (12-U-R-4) and (12-U-L-4). Then the gear teeth (14) of the mini rollers (12-U-L-3) and (12-U-L-4) pull the next portion of upper sting (5-U) and the lower string (5-L) respectively and in turn rotates mini rollers of (12-U-R-2) and (12-U-R-5) respectively. By mechanism described above the tension of string (5) applied on the string is transferred to the remote portion of the string (5) that locates between the mini rollers of (12-U-L-1) and (12-U-L-6) with minimum loss of tension.

In the frame of the applicant's previous sample described in the U.S. Pat. No. 7,905,849 the rollers provide friction when the frame is tighten hardly. And as a result, the frame is not fastened uniformly throughout the frame.

Meanwhile, like a chain and gear of a bicycle, the gear teeth (14) of the mini roller (12) according to current application transfers tension to the next mini roller with minimum loss of tension. As a result, the wearer feels comfortable because each portion of the back brace frame presses the body portion with same pressure.

1. A waist support frame for a detachment type waist protecting belt to hold the vertebra region of a vertebra related patient, whose one hand is not available, without rotating the frame around the user's body with only one hand is comprised of;
two back rest frames,
and
one power module, which is comprised of two back brace plates, each of which is equipped with four pairs of pulling code conveying mini rollers, which has pluralities of gear teeth developed on the surface thereof along the vertical axis,
and
one string that goes around 8 pairs of pulling code conveying mini rollers and 2 remote mini rollers,
and
one puller that fasten the two back rest frames,
and
two set of broad bands.

2. A waist support frame for a detachment type waist protecting belt to hold the vertebra region of a vertebra related patient, whose one hand is not available, without rotating the flame around the user's body with only one hand of the claim 1, wherein upper corner and lower corner of the mini roller are rounded.
